(19) United States
(12) Patent Application Publication
(10) Pub. No.: US 2014/0322744 A1
(43) Pub. Date: Oct. 30, 2014

(54) CLONABLE TAG FOR CORRELATIVE LIGHT AND ELECTRON MICROSCOPY LABELING

(71) Applicants: INSTITUT CURIE, PARIS CEDEX 05 (FR); INSERM (INSTITUT NATIONAL DE LA SANTE ET DE LA RECHERCHE MEDICALE), PARIS (FR); CENTRE NATIONAL DE LA RECHERCHE SCIENTIQUE, PARIS (FR)

(72) Inventors: Nebraska Zambrano, Reims (FR); Vincent Semetey, Le Mans (FR); Sergio Marco, Saint Germain En Laye (FR)

(73) Assignees: Institut Curie, Paris (FR); Inserm (Institut National de la Sante et de la Recherche Medicale), Paris (FR); Centre National de la Recherche Scientifique, Paris (FR)

(21) Appl. No.: 14/359,947

(22) PCT Filed: Nov. 27, 2012
(86) PCT No.: PCT/EP2012/073659

§ 371 Date: May 22, 2014

(30) Foreign Application Priority Data
Nov. 28, 2011 (EP) 11306572.6

(51) Int. Cl. C12Q 1/02

(52) U.S. Cl. CPC C12Q 1/02
USPC 435/29; 530/324; 530/350; 435/320.1; 435/252.33

(57) ABSTRACT

The present invention relates to a new clonable label for Correlative Light and Electron Microscopy based on the combination of modified metallothionein and lanthanides.

### FIELD OF THE INVENTION

The present invention relates to a label for microscopy, in particular for light and electron correlative microscopy (CLEM).

### BACKGROUND OF THE INVENTION

Biological mesoscale structures are mainly imaged by light microscopy (LM) and by transmission electron microscopy (TEM). These approaches are complementary with others such as nuclear magnetic resonance (NMR) or X-ray crystallography providing information about sizes, dynamics, localizations, and arrangements of sub-cellular components within their cellular context. This complementarity has led researchers to develop correlative imaging (CI). CI consists to combine data obtained, by several techniques at different resolutions, from a single material. Because of the technical difficulties to perform CI with a large number of imaging methods, scientists have concentrated their efforts in correlating data from LM and TEM. Both imaging methods contained the most global ranges of values of resolution interesting cell biology. Thus, LM allows the observation of objects from micrometer until millimeter whereas TEM provides data access from nanometers to micrometers. Combining both methods TEM and LM for a correlative imaging is called correlative light and electron microscopy (CLEM).

CLEM has greatly contributed to the functional understanding of complex biological systems. The application of correlated LM and TEM is leading the way in revolutionizing the understanding of the structural basis of cell biology. Developments in CLEM probes provide great opportunities to determine protein co-localization, turnover, and function. The difficulty of each CLEM study depends on the type of sample and on the questions to be answered. For instance, if a protein should be located, a specific labeled probe valuable for different detection methods is required. Often, the probes for LM and TEM are different: LM gives preference to fluorescent probes which are markers coupled with the studied protein whereas TEM requires electron dense particles which are mainly gold-antibodies. In addition, when probes are not clonable, some artifacts can be induced because of the sample preparation methods such as permeabilization or use of indirect detection approaches (i.e. antibodies). This raises the question about how to perform to improve correlative imaging. Nowadays a major challenge in this field is the development of clonable probes which are simultaneously fluorescent and electron dense, thus facilitating CLEM.

One way of highlighting proteins of interest within cells is to attach labels to them. For LM, this involves frequently the tagging of molecules with fluorophores. However, TEM markers must be highly electron dense in order to be distinguished from background. These TEM markers are non fluorescent heavy metals (Au, Hg, Cd, U, etc.). Probes for CLEM should combine fluorescence and electron density properties. Today, the LM and TEM probes, which can be adapted for CLEM, can be grouped in two categories: precipitation methods (fluorescence-photo-oxidation or enzymatic approaches), and nanoparticles (Quantum dots). Colloidal gold antibodies and Tag-labels are frequently used for TEM and LM, but in non correlative approaches.

The fluorescence-photo-oxidation is described for high-resolution light and electron microscopy immune-localization of proteins in cells and tissues. It implies immunofluorescence and subsequent photo-oxidation of several reactants into an insoluble osmiophilic polymer. This method presents the following disadvantages. Photo-conversion of some chemicals, such as diaminobenzidine (DAB), also occurs in mitochondria, thereby contributing to nonspecific background in the sample. Therefore, chemicals need to be changed every few minutes in order to prevent oxidation. During this process, the fluorescence fades quickly and brownish reaction product began to appear in place of fluorescence. After photo-oxidation, a fixation is necessary and samples slices are dehydrated. Permeabilization step is required, which can induce artifacts, and sample preparation is long.

Enzyme-based methods are straightforward and cheap; they penetrate well into the sample. However, similarly to photo-oxidation, the time preparation is long and the protocol needs to be adapted for each sample. In addition, this method presents other disadvantages such as diffusion of the reaction products away from the reaction site and onto membranous structures which decrease the resolution of the labeling. Also, a permeabilization step is required. To observe sample in TEM, permeabilization and resin inclusion is necessary. Several steps of staining are necessary. Finally, sample preparation is long, and unspecific reaction of antibodies can occur.

Quantum dots are inorganic nano-crystals (4-10 nm) that can be excited with a single electron and re-emit light in a very specific wavelength depending on their size. In consequence, these inorganic nano-crystals fluoresce at distinct wavelengths depending on their size and shape. The core, typically a CdSe or CdTe crystal, are electron dense and enables discrimination of the distinct QDs at the EM level. In spite of their interest, this method presents important disadvantages, mainly because of a permeabilization step is required and because of the small size of quantum dots, making difficult TEM imaging.

Both precipitation and nanoparticles approaches need specific adaptation for each sample inducing a poor reproducibility of results and reducing their accuracy. Thus, there is still a strong need of probes which are simultaneously fluorescent and electron dense for CLEM. Such probes should avoid the preparation of two distinct samples, one for TEM and the other for LM, which is time consuming and male the analysis more difficult. Moreover, ideal probes should be clonable to prevent artifacts and to warranty specificity. For light microscopy, clonable reagents such as green fluorescent protein have represented a real revolution and equivalent reagents for transmission electron microscopy have been pursued for a long time. WO2006/118615 describes the use of metallothionein clonable tags for electron microscopy using Au, Ag, Hg, Cd, Zn, Pt or Bi. However, those atoms are not fluorescent and can be only used for electron microscopy. Therefore, the known clonable tags are either suitable for light microscopy or for electron microscopy.

Finally, valuable probes should prevent the artifacts due to permeabilization or the photo-oxidation.

### SUMMARY OF THE INVENTION

The present invention provides an interesting probe suitable for correlative electron and light microscopy and meeting the above-mentioned advantages, by using lanthanides, in particular Europium, in combination with mutated Metallothionein-tags (MTtag).

From a biological point of view, lanthanides are not naturally present in organisms and have no known biological role. Therefore there is no intrinsic expression of lanthanides in a biological environment. Thus, their detection necessarily results from its introduction during the experiment, limiting artifacts. From a methodological point of view, lanthanides present interesting electron dense and fluorescence properties. These two characteristics allow lanthanides to be tracked by electron microscopy and light microscopy. However, the use of lanthanides as probe needs the amplification of their intrinsic fluorescence. In addition, they should be targeted to the protein of interest.

By modifying a metallothionein protein domain to render it suitable for transferring energy to lanthanides, the present invention provides a convenient clonable tag suitable for both electron microscopy and light microscopy methods.

Therefore, the present invention relates to a metallothionein tag, preferentially able to sequester lanthanides, comprising a metallothionein or a fragment thereof of at least 60 amino acids including the cysteine-rich domain, wherein the metallothionein tag has at least one substitution by a tryptophan residue of an amino acid directly adjacent to a cysteine residue, preferably directly surrounded by two cysteine residues. More preferably, the metallothionein tag comprises or consists of the sequence of SEQ ID No 1, 2 or 3 and has at least one substitution by a tryptophan residue of an amino acid in position selected from the group consisting of 4, 6, 8, 12, 14, 16, 18, 20, 22, 23, 25, 27, 28, 30, 32, 35, 38, 40, 42, 43, 45, 47, 49, 51, 56, 58 and 61, more preferably selected from the group consisting of 6, 14, 20, 25, 35, 49 and 58. In particular, the residue in position 49 has been substituted by a tryptophan residue and, optionally one or several amino acids in positions 4, 6, 8, 12, 14, 16, 18, 20, 22, 23, 25, 27, 28, 30, 32, 35, 38, 40, 42, 43, 45, 47, 51, 56, 58 and 61, preferably in positions 6, 14, 20, 25, 35 and 58, have also been substituted by a Tryptophan residue.

The present invention further relates to a protein or polypeptide comprising at least one metallothionein tag according to the invention and a protein of interest, preferably as a fusion protein.

The present invention also relates to a protein-lanthanide complex comprising
1. a metallothionein tag according to the invention or a protein or polypeptide comprising at least one metallothionein tag according to the invention and a protein of interest, preferably as a fusion protein and,
2. a lanthanide bound to the metallothionein tag, preferably selected from the group consisting of Eu (Europium), Terbium (Tb), Samarium (Sm) and Dysprosium (Dy), more preferably Eu (Europium).

The present invention relates to a nucleic acid, expression construct or expression vector encoding a metallothionein tag according to the present invention or a protein or polypeptide comprising at least one metallothionein tag according to the invention and a protein of interest, preferably as a fusion protein. In a particular embodiment, the nucleic acid, expression construct or expression vector comprises a nucleic acid encoding a metallothionein tag according to the present invention and a polylinker upstream or downstream of a nucleic acid encoding a metallothionein tag.

The present invention relates to a recombinant cell comprising the nucleic acid, expression construct or expression vector of the invention.

It also relates to a kit comprising the nucleic acid, expression construct or expression vector comprising a nucleic acid encoding a metallothionein tag according to the present invention and a polylinker upstream or downstream of a nucleic acid encoding a metallothionein tag, and optionally other reagents such as a lanthanide and/or a restriction enzyme.

In addition, the present invention relates to the use of a metallothionein tag according to the invention, a protein or polypeptide comprising at least one metallothionein tag according to the invention and a protein of interest, preferably as a fusion protein, a complex as disclosed above, a nucleic acid, expression construct or expression vector as disclosed above, a recombinant cell as disclosed above, or a kit as disclosed above for microscopy analysis, preferably for both electron and light microscopy analysis.

Finally, the present invention relates to a method for analyzing a protein of interest by light and/or electron microscopy, comprising
a. providing a sample comprising a fusion protein according to the present invention;
b. contacting the sample with a lanthanide; and,
c. analyzing the sample by light and/or electron microscopy.

Preferably, the fusion protein is present in a cell. Optionally, the step a) comprises providing a cell comprising a nucleic acid, expression construct or expression vector encoding a fusion protein according to the present invention, and cultivating the cell under conditions allowing the expression of the fusion protein. Preferably, the lanthanide is selected from the group consisting of Eu (Europium), Terbium (Tb), Samarium (Sm) and Dysprosium (Dy), preferably Eu (Europium). Optionally, the sample is prepared for being suitable for microscopy analysis prior to the analyzing step.

### DESCRIPTION OF THE DRAWINGS

FIG. 1: E. coli K-12 MG1655 growth curve in the presence of several concentrations of Europium. Graphics suggest that Europium doesn't have a dramatic negative effect on bacterial growth.

FIG. 2: MT-Eu Interaction. FIG. 2A) EuCl₃ MIC of E. coli MG1655 without MT;

FIG. 2B) EuCl₃ MIC of E. coli MG1655 with MT. The curves suggest an interaction between MTtag and Eu³⁺ on cell. Normalized growth value=(DO_{550 nm total} growth of sample with Eu X concentration)/(DO_{550 nm total} growth of sample with Eu 0 μM)

FIG. 3: Comparative for specific intracellular molecular tracking by TEM and EELS. (1) Cells expressing MT treated with EuCl₃ and induced with 0.01% arabinose a) STEM-HAADF image; b) TEM image that electron dense nanometric particles are only visible in bacteria expressing MTtag growing in presence of EuCl₃; c) EELS spectrum of sample. (2) Images of no transformed cells treated with EuCl₃; a) STEM-HAADF; b) TEM; and c) EELS; suggest that nano cluster inner cells are only present when MTtag is present. (3) Europium crystal a) STEM-HAADF; b) TEM; c) EELS spectrum.

FIG. 4: FIG. 4A) Scheme of pMAL™-c5X allowing expressing MBP-MTtag fusion. Cloning is done by restriction directly downstream of the specific protease site. FIG. 4B) DNA checking in a 0.8% agarose gel. DNA digestion with XmnI/PstI restriction enzymes. Results suggest a correct cloning of MT into the vector.

FIG. 5: MBP-MTtag purification protein SDS gel. Purification of fusion protein was obtained in elution fraction 5-12. A single band ˜56 KDa correspond to fusion size was observed on elution fractions, suggest a pure extraction.

FIG. 6: Emission Scan by MTtag-Eu complex. Spectrum of dialyzed protein MTtag mixed with 200 mM EuCl₃ showed that, no fluorescence is present at 280 nm excitation, however signals are found when the excitation wavelength is 393 nm, i.e, when Eu³⁺ is present.

FIG. 7: Antenna effect: The energy absorbed for antenna is transmitted on lanthanide, making it able to emit.

FIG. 8: Dialyzed EuCl₃ spectrums. Typical Eu³⁻ signal does not present.

FIG. 9: Emission spectrum of Purified MT Eu-Binding. The emission spectrum of MT wt, mutant (50 nM) and buffer control, in the presence of Eu³ (200 nM) with excitation at 280 nm.

FIG. 10: Tracking of MBP-MT49Eu³⁺ fusion proteins expressed on pMALc5x (1a, 1b and 2a) and pMALp5X (2b) vectors growth in presence of 1 mM of EuCl₃. Intracellular cytoplasm localization by fusion expressed in a pMALc5x is confirmed in 1 by HAABF (a) and DF STEM and (2a) TEM mode. Membrane localization is show by fusion protein expressed in a pMaLp5x by Tomo-TEM (2b). 3) Nano cluster were not present on non transformed cells a) HAABF; b) DF STEM mode. Sacle bar: 1 and 3 (a,b) 500 nm; 2 (a,b) 100 nm.

### DETAILED DESCRIPTION OF THE INVENTION

Abbreviation

CLEM: Correlative Light and electron microscopy

GFP: Green Fluorescent Protein

LM: Light Microscopy

MBP: Maltose Binding Protein

MIC: Minimal Inhibitory Concentration

MT: Metallothionein

TEM: Transmission Electron Microscopy

SEM: Scanning Electron Microscopy

The inventors have worked to develop a simple LM and TEM correlative probe allowing the visualization of proteins in living cells and the study of their intracellular dynamics in resin embedded and frozen-hydrated samples. The localization of proteins in a single sample can reveal new relationships between cell ultra-structure, and protein distribution and dynamics related with metabolism.

Most performing current techniques for TEM-LM correlative imaging are technically complex, requires special equipments and needs long time periods. A sample preparation needs more than 7 days when people with good technical skills and experience are involved and, in some cases, implies staining, permeabilization and use of different antibodies leading to artifacts and low accuracy on the target protein localization. The best way to overcome these limits is the use of small clonable fluorescence and electron dense fusion-proteins acting such as GFP but suitable for TEM.

The advantages of a fusion tag with these characteristics are the following:
Sample preparation becomes easier because the internal expression eliminates permeabilization steps;
The tag signal is directly associated with the target protein, increasing accuracy on the localization;
The expression level of fused protein can be controlled, allowing the study; and
Standard molecular biology methods can be used and therefore special training or high technical skills are not required.

In order to design a probe having the described characteristics, the tag should be photoluminescent and electron-dense. Interestingly, lanthanides have both properties but they are not clonable.

The present work is based on the combination of metallothionein (MT) used as clonable tags with lanthanides to produce a modified MT-tag florescent and electron dense probe.

Lanthanides

Lanthanides (Ln) are rare earth elements (or rare earth metals) including the elements with atomic numbers from 57 to 71 but also, Ytrium (atomic no. 39) and Scandium (atomic no. 21). The principal source of rare earths elements is the mineral monazite. The f-electrons transitions in Ln are responsible for their fluorescence properties.

The f transition implies energy changes in the atom, producing spectral holes and inducing luminescence. The f-f electronic transitions in Lns lead to long excited state lifetimes, from the microsecond to millisecond range. In addition, its low extinction coefficients in Ln is due to the high energy necessary for f-f transitions, making direct photoexcitation difficult, i.e. Ln by itself has poor ability to absorb a photon of energy and excite his f-electrons. Ln can form complexes with various organic molecules such as beta-diketones, polyaminopolycarboxylic acids (EDTA), β-mercaptoethanol, (poly)pyridines and calixarenes. When the ligands contain organic chromophores with suitable photophysical properties, highly luminescent Ln complexes can be obtained.

The trivalent cations of the Ln have photoluminescent properties that are favorable for several kinds of applications. However, it is difficult to generate this luminescence by direct excitation of the Ln ion, because of the poor ability of ions to absorb light. However, organic chromophores can absorb the energy and transfer it to a nearby Ln ion, which is then able to emit its characteristic luminescence.

Europium is one of the most interesting lanthanide for luminescent studies. It has a red emission line at 612 nm. The main characteristics of Eu can be summarized as follows:
It is a soft silvery metal and the most reactive lanthanide;
The energy space between the most emissive level and the highest sub-level of the ground state is large enough to almost avoid autofluorescence but, at high concentrations (higher than 10 mM), it can be detected by standard LM;
Eu high-energy in photons is operative and its luminescence quenching can be turned into an advantage for the determination of the hydration number of the sample;
The lifetime of the fluorescence is in few millisecond range and may even reach values up depending on the associate chromophore;
It is an inorganic element, thus allowing time-resolved detection with simple instrumentation, a definite advantage for analytical applications;
Depending on how the Eu is incorporated in the chromophore, the emission can be almost monochromatic. In addition, the emission color can be adjusted by reversing the magnetic dipole center transition element and promoting electric-type transitions;
Eu ions are very stable, because it is originated from internal transitions and therefore, no chemical or physical bonds are involved;
It has a high resistance to photo-bleaching and an exceptional resistance to photo and chemical degradation;
Eu salts are soluble in water (e.g., EuCl₃);
From a biological point of view, Eu has no known biological role. Eu-based nanoparticles has been described and mainly localized in cytoplasmic compartments with no apparent effect on the eukaryotic cell viability;
Eu toxicity has not been reported; and
Eu-based nanoparticles are used for diverse biomedical applications.

Lanthanides, other than Europium, (e.g., Tb, Sm, Nd, Tm, Ho, Ce and Dy) may also be used in the present invention. More particularly, the following lanthanides (non-exhaustive list thereof) can be used: Terbium (Tb), Tamarium (Sm) and Dysprosium (Dy). The emission peaks of Eu, Sm, Tb, and Dy complexes are 615 nm, 643 nm, 545 nm, and 574 nm, respectively.

Metallothionein

Metallothionein (MT) is a family of cysteine-rich, low molecular weight proteins. Metallothioneins form a super family of proteins found on different organism sources: Eukaryota; Metazoa; Chordata; Craniata; Vertebrata; Chondrichthyes; Elasmobranchii; Galeomorphii; Galeoidea; Carcharhiniformes; Scyliorhinidae; Scyliorhinus. The MT consensus includes 60 amino acids with a high content of Cysteine (Cys) residues that bind various heavy metals. In addition to binding the heavy metals, metallothioneins present the property to be able to bind lanthanides. Metallothionein proteins have been cloned from a wide variety of organisms and their sequences may be found in various publicly available databases such as GenBank (world wide web at ncbi.nkn.nih.gov). Exemplary metallothionein genes include, for example, those from human (GenBank Accession No. NM_{—}005946 (nucleotide), NP_{—}005937 (protein)); mouse (GenBank Accession No. NM_{—}013602 (nucleotide), NP_{—}038630 (protein)); rat (GenBank Accession No. NM_{—}138826 (nucleotide), NP_{—}620181 (protein)); and rabbit (GenBank Accession No. X07791 (nucleotide), S54334 (protein)).

Such a consensus sequence may be defined as SEQ ID Nos 1, 2 or 3:

(SEQ ID No 1)
(M/abs)-D-(P/L)-(N/S/D/T)-C-(S/F)-C-(S/A/T/I/P/V/
E)-(T/S/A/P)-(G/D)-(G/D/C/V/E/N)-S-C-(T/A/S/P/M/
D)-C-(T/A/P/S/G)-(S/E/G/T/D/N/R)-S-C-(A/N/K/T/G)-
C-K-(N/E/D/A/Q)-C-(K/R)-C-(T/A/P)-S-C-K-K-(S/N/I)-
C-C-(S/P/F/A/T)-C-C-P-(V/A/M/R/L)-G-C-(S/A/T)-(K/
R)-C-A-Q-G-C-(V/I)-C-K-(G/E)-(A/T/M/S/E)-(A/L/S/
T)-(D/E)-K-C-(T/S/C/N/R/I)-C-C-(A/abs)
(SEQ ID No 2)
(M/abs)-D-P-N-C-S-C-(S/A/T)-T-G-G-S-C-T-C-(T/A)-
(S/G)-S-C-(A/K/T)-C-K-(N/E)-C-K-C-T-S-C-K-K-S-
C-C-S-C-C-P-V-G-C-(S/A)-K-C-A-Q-G-C-(V/I)-C-K-G-A-
(A/S)-(D/E)-K-C-(T/S)-C-C-(A/abs)
(SEQ ID No 3)
MDPNCSCSTG GSCTCTSSCA CKNCKCTSCK KSCCSCCPVG
CSKCAQGCVC KGAADKCTCC A

The inventors propose to use the ability of the metallothionein to bind lanthanides and to modify metallothionein in order to render it suitable as antenna for lanthanides. Therefore, the metallothionein is modified by substituting amino acid(s) by tryptophan residue(s) in position as close as possible of the binding site of lanthanides for allowing energy transfer between tryptophan and lanthanides. Cysteine-rich domain, and more particularly cysteine residues, being responsible of the lanthanides binding, the inventors define that the substitution has to take place in the direct surrounding of cysteine residues. According, one or several residues directly adjacent to a cysteine residue are substituted by a tryptophan residue (C-X or X-C). Preferably, the inventors defined that the most suitable sites are the residues which are surrounded by two cysteine residues (C-X-C).

Accordingly, the metallothionein tag of the invention comprises or consists of a metallothionein or a fragment thereof of at least 60 amino acids including the cysteine-rich domain, wherein the metallothionein tag has at least one substitution by a tryptophan residue of an amino acid directly adjacent to a cysteine residue, preferably an amino acid directly surrounded by two cysteine residues.

More specifically, in consensus sequences defined in SEQ ID Nos 1, 2 and 3, one or several amino acids in positions 4, 6, 8, 12, 14, 16, 18, 20, 22, 23, 25, 27, 28, 30, 32, 35, 38, 40, 42, 43, 45, 47, 49, 51, 56, 58 and 61 are substituted by Tryptophan, i.e. in positions underlined below.

(SEQ ID No 1)
(M/abs)-D-(P/L)-(N/S/D/T)-C-(S/F)-C-(S/A/T/I/P/
V/E)-(T/S/A/P)-(G/D)-(G/D/C/V/E/N)-S-C-(T/A/S/
P/M/D)-C-(T/A/P/S/G)-(S/E/G/T/D/N/R)-S-C-(A/N/K/
T/G)-C-K-(N/E/D/A/Q)-C-(K/R)-C-(T/A/P)-S-C-K-K-
(S/N/I)-C-C(S/P/F/A/T)-C-C-P-(V/A/M/R/L)-G-C-
(S/A/T)-(K/R)-C-A-Q-G-C-(V/I)-C-K-(G/E)-(A/T/M/
S/E)-(A/L/S/T)-(D/E)-K-C-(T/S/C/N/R/I)-C-C-A
(SEQ ID No 2)
(M/abs)-D-P-N-C-S-C-(S/A/T)-T-G-G-S-C-T-C-(T/A)-
(S/G)-S-C-(A/K/T)-C-K-(N/E)-C-K-C-T-S-C-K-K-S-C-C-
S-C-C-P-V-G-C-(S/A)-K-C-A-Q-G-C-(V/I)-C-K-G-A-
(A/S)-(D/E)-K-C-(T/S)-C-C-A
(SEQ ID No 3)
(M/abs)-DPNCSCSTGG SCTCTSSCAC KNCKCTSCKK SCCSCCPVGC
SKCAQGCVCK GAADKCTCCA

In a preferred embodiment, in consensus sequences defined in SEQ ID Nos 1, 2 and 3, one or several amino acids in positions 6, 14, 20, 25, 35, 49 and 58 are substituted by Tryptophan, i.e. in positions underlined below.

(SEQ ID No 1)
(M/abs)-D-(P/L)-(N/S/D/T)-C-(S/F)-C-(S/A/T/I/P/
V/E)-(T/S/A/P)-(G/D)-(G/D/C/V/E/N)-S-C-(T/A/S/
PM/D)-C-(T/A/P/S/G)-(S/E/G/T/D/N/R)-S-C-(A/N/K/
T/G)-C-K-(N/E/D/A/Q)-C-(K/R)-C-(T/A/P)-S-C-K-K-
(S/N/I)-C-C-(S/P/F/A/T)-C-C-P-(V/A/M/R/L)-G-C-
(S/A/T)-(K/R)-C-A-Q-G-C-(V/I)-C-K-(G/E)-(A/T/M/
S/E)-(A/L/S/T)-(D/E)-K-C-(T/S/C/N/R/I)-C-C-A
(SEQ ID No 2)
(M/abs)-D-P-N-C-S-C-(S/A/T)-T-G-G-S-C-T-C-(T/A)-
(S/G)-S-C-(A/K/T)-C-K-(N/E)-C-K-C-T-S-C-K-K-S-C-
C-S-C-C-P-V-G-C-(S/A)-K-C-A-Q-G-C-(V/I)-C-K-G-A-
(A/S)-(D/E)-K-C-(T/S)-C-C-A
(SEQ ID No 3)
MDPNCSCSTGG SCTCTSSCAC KNCKCTSCKK SCCSCCPVGC
SKCAQGCVCK GAADKCTCCA

Accordingly, the metallothionein tag of the invention comprises or consists of a sequence of SEQ ID No 1, 2 or 3, wherein one or several amino acids in positions 4, 6, 8, 12, 14, 16, 18, 20, 22, 23, 25, 27, 28, 30, 32, 35, 38, 40, 42, 43, 45, 47, 49, 51, 56, 58 and 61, preferably in positions 6, 14, 20, 25, 35, 49 and 58, have been substituted by a Tryptophan residue.

In a first particular embodiment, the metallothionein tag of the invention comprises or consists of a sequence of SEQ ID No 1, 2 or 3, wherein one amino acid in position 4, 6, 8, 12, 14, 16, 18, 20, 22, 23, 25, 27, 28, 30, 32, 35, 38, 40, 42, 43, 45, 47, 49, 51, 56, 58 or 61, preferably in position 6, 14, 20, 25, 35, 49 or 58, has been substituted by a Tryptophan residue. Preferably, the residue in position 49 has been substituted by a tryptophan residue.

In a second particular embodiment, the metallothionein tag of the invention comprises or consists of a sequence of SEQ ID No 1, 2 or 3, wherein several amino acids in positions 4, 6, 8, 12, 14, 16, 18, 20, 22, 23, 25, 27, 28, 30, 32, 35, 38, 40, 42, 43, 45, 47, 49, 51, 56, 58 and 61, preferably in positions 6, 14, 20, 25, 35, 49 and 58, have been substituted by a Tryptophan residue. For instance, the metallothionein tag of the invention may include 2, 3, 4, 5 or 6 substitutions of a residue by Tryptophan.

Accordingly, in particular embodiment, tryptophan residues may be substituted at the positions: 6 and 14; 6 and 14; 6 and 20; 6 and 25; 6 and 35; 6 and 49; 6 and 58; 14 and 20; 14 and 25; 14 and 35; 14 and 49; 14 and 58; 20 and 25; 20 and 35; 20 and 49; 20 and 58; 25 and 35; 25 and 49; 25 and 58; 35 and 49; 35 and 58; 49 and 58; 6, 14 and 20; 6, 14 and 25; 6, 14 and 35; 6, 14 and 49; 6, 14 and 58; 6, 20 and 25; 6, 20 and 35; 6, 20 and 49; 6, 20 and 58;6, 25 and 35; 6, 25 and 49; 6, 25 and 58;6, 35 and 49; 6, 35 and 58; 6, 49 and 58; 14, 20 and 25; 14, 20 and 35; 14, 20 and 49; 14, 20 and 58; 14, 25 and 35; 14, 25 and 49; 14, 25 and 58; 14, 35 and 49; 14, 35 and 58; 14, 49 and 58; 20, 25 and 35; 20, 25 and 49; 20, 25 and 58; 20, 35 and 49; 20, 35 and 58; 20, 49 and 58; 25, 35 and 49; 25, 35 and 58; 25, 49 and 58; 35, 49 and 58; 6, 14, 20 and 25; 6, 14, 20 and 35; 6, 14, 20 and 49; 6, 14, 20 and 58; 6, 14, 25 and 35; 6, 14, 25 and 49; 6, 14, 25 and 58; 6, 14, 35 and 49; 6, 14, 35 and 58; 6, 14, 49 and 58; 6, 20, 25 and 35; 6, 20, 25 and 49; 6, 20, 25 and 58; 6, 20, 35 and 49; 6, 20, 35 and 58; 6, 20, 49 and 58; 6, 25, 35 and 49; 6, 25, 35 and 58; 6, 25, 49 and 58; 6, 35, 49 and 58; 14, 20, 25 and 35; 14, 20, 25 and 49; 14, 20, 25 and 58; 14, 20, 35 and 49; 14, 20, 35 and 58; 14, 25, 35 and 49; 14, 25, 35 and 58; 14, 25, 49 and 58; 14, 35, 49 and 58; 20, 25, 35 and 49; 20, 25, 35 and 58; 20, 25, 49 and 58; 25, 35, 49 and 58; 6, 14, 20, 25 and 35; 6, 14, 20, 25 and 49; 6, 14, 20, 25 and 58; 6, 14, 25, 35 and 49; 6, 14, 25, 35 and 58; 6, 14, 35, 49 and 58; 6, 20, 25, 35 and 49; 6, 20, 25, 35 and 58; 6, 20, 35, 49 and 58; 6, 25, 35, 49 and 58; 14, 20, 25, 35 and 49; 14, 20, 25, 35 and 58; 14, 20, 35, 49 and 58; 14, 25, 35, 49 and 58; 6, 14, 20, 25, 35 and 49; 6, 14, 20, 25, 35 and 58; 6, 14, 20, 25, 49 and 58; 6, 14, 20, 35, 49 and 58; 6, 14, 25, 35, 49 and 58; 6, 20, 25, 35 and 49; 14, 20, 25, 35, 49 and 58; or 6, 14, 20, 25, 35, 49 and 58.

Preferably, the residue in position 49 has been substituted by a tryptophan residue and one or several amino acids in positions 4, 6, 8, 12, 14, 16, 18, 20, 22, 23, 25, 27, 28, 30, 32, 35, 38, 40, 42, 43, 45, 47, 51, 56, 58 and 61, preferably in positions 6, 14, 20, 25, 35 and 58, have also been substituted by a Tryptophan residue. Accordingly, in particular embodiment, tryptophan residue may be substituted at the positions: 6 and 49; 14 and 49; 20 and 49; 25 and 49; 35 and 49; 49 and 58; 6, 14 and 49; 6, 25 and 49; 6, 35 and 49; 6, 49 and 58; 14, 20 and 49; 14, 25 and 49; 14, 35 and 49; 14, 49 and 58; 20, 25 and 49; 20, 35 and 49; 20, 49 and 58; 25, 35 and 49; 25, 49 and 58; 35, 49 and 58; 6, 14, 20 and 49; 6, 14, 25 and 49; 6, 14, 35 and 49; 6, 14, 49 and 58; 6, 20, 25 and 49; 6, 20, 35 and 49; 6, 20, 49 and 58; 6, 25, 35 and 49; 6, 25, 49 and 58; 6, 35, 49 and 58; 14, 20, 25 and 49; 14, 20, 35 and 49; 14, 25, 35 and 49; 14, 25, 49 and 58; 14, 35, 49 and 58; 20, 25, 35 and 49; 20, 25, 49 and 58; 25, 35, 49 and 58; 6, 14, 20, 25 and 49; 6, 14, 25, 35 and 49; 6, 14, 35, 49 and 58; 6, 20, 25, 35 and 49; 6, 20, 35, 49 and 58; 6, 25, 35, 49 and 58; 14, 20, 25, 35 and 49; 14, 20, 35, 49 and 58; 14, 25, 35, 49 and 58; 6, 14, 20, 25, 35 and 49; 6, 14, 20, 25, 49 and 58; 6, 14, 20, 35, 49 and 58; 6, 14, 25, 35, 49 and 58; 6, 20, 25, 35 and 49; 14, 20, 25, 35, 49 and 58; or 6, 14, 20, 25, 35, 49 and 58.

In a very particular embodiment, the present invention relates to a metallothionein tag comprising a sequence of SEQ ID No 1, 2 or 3, wherein the amino residue at position 49 has been substituted by a tryptophan.

The present invention further relates to a metallothionein tag, which presents at least 90, 95, 96, 97 or 98% of identity with the metallothionein tags as disclosed above, provided that none of the cysteine residues and the substituting tryptophan(s) is changed.

The percentage of sequence identity between two biological sequences can be determined by comparing two optimally aligned sequences over a window of comparison of, for example, at least 20 positions. Preferably, the percent identity is determined over two sequences of identical size. The percentage is calculated by determining the number of positions at which the identical amino acid residue occurs in both sequences to yield the number of matched positions, dividing the number of matched positions by the total number of positions in the window of comparison (i.e., the window size), and multiplying the result by 100 to yield the percentage of sequence identity. Optimal alignment of sequences for comparison may be conducted by the local homology algorithm of Smith and Waterman, Adv. Appl. Math., 2:482 (1981), by the homology alignment algorithm of Needleman and Wunsch, J. Mol. Biol., 48:443 (1970), by the search for similarity method of Pearson and Lipman, Proc. Natl. Acad. Sci. (U.S.A.), 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. These references are incorporated herein by reference.

The present invention also relates to a polypeptide or protein comprising several metallothionein tags of the invention. For instance, it can include 2, 3, 4, 5 or 6 metallothionein tags.

The present invention also relates to a polypeptide or protein comprising a metallothionein tag of the invention and a purification tag, such as a His tag (His₆), a FLAG tag, a HA tag (epitope derived from the Influenza protein haemagglutinin), a MYC tag (epitope derived from the human proto-oncoprotein MYC) or a GST tag (small glutathione-S-transferase). Preferably, the purification tag is at the N-terminal end or C-terminal end of the protein, but it also can be placed in any other position of the targeted protein, mainly in the case of proteins composed by several domains.

The present invention further relates to a polypeptide or protein, also referred as fusion protein, comprising at least one metallothionein tag of the invention fused directly or via a linker, preferably a peptidic linker, to a protein or polypeptide of interest. For instance, the linker may be a short peptide of 1-50, 2-20 or 2-10 amino acids. In particular, the protein or polypeptide of interest is the protein to be studied and visualized in microscopy. The metallothionein tag(s) (MT tag) may be at the N-terminal end or C-terminal end of the protein or polypeptide of interest or both at the N-terminal and C-terminal ends of the protein or polypeptide of interest. Optionally, the polypeptide or protein may further comprise a purification tag.

The present invention finally relates to lanthanides bound to a polypeptide or protein as described above, also referred as a complex lanthanide-MT tag. Preferably, the lanthanide is selected from the group consisting of Eu, Sm, Tb, and Dy. More preferably, the lanthanide is Europium. The lanthanides are bound to the metallothionein tag.

Nucleic Acid and Vectors

The present invention also relates to a nucleic acid encoding a metallothionein tag as disclosed above or a polypeptide or protein, in particular a fusion protein, comprising at least one metallothionein tag of the invention and the protein or polypeptide of interest.

In the present invention, the term “nucleic acid” shall be understood to mean a polymer of nucleotides, including DNA or RNA, either single- or double-stranded. These can be molecules that are synthetic or semi-synthetic, recombinant, optionally amplified or cloned, chemically modified or containing non-natural bases. The phrase “nucleic acid sequence” or “nucleotide sequence” refers to a single- or double-stranded polymer of deoxyribonucleotide or ribonucleotide bases. The term “isolated” when referring to nucleic acids, refer to those that have been purified away from other cellular components and contaminants, i.e., other cellular nucleic acids and/or proteins, by standard techniques, including, for example, alkaline/SDS treatment, CsCl banding, column chromatography, and others purification techniques well known in the art. See, e.g., Methods in Enzymology, Vol. 152: Guide to Molecular Cloning Techniques (Berger and Kimmel (eds.), San Diego: Academic Press, Inc. (1987)), and Current Protocols in Molecular Biology (Ausubel, et al., (ed.), Greene Publishing and Wiley-Interscience, New York (1987)), both of which are incorporated herein by reference.

For instance, such a sequence encoding the metallothionein tag of SEQ ID No 3 is the following:

(SEQ ID No 4)
ATG GAC CCG AAT TGT ACG TGT AGC ACC GGT GGT AGC
TGT ACC TGT ACC AGC TCT TGT GCA TGT AAA AAT TGT
AAA TGC ACC AGC TGC AAA AAA AGC TGT TGT AGC TGT
TGC CCG GTT GGT TGT AGC AAA TGT GCA CAG GGT TGT
GTT TGT AAA GGT GCA GCA GAT AAA TGT ACC TGT TGT
GCA GGA TCC TAA

In order to introduce the tryptophan substitution, the sequence is mutated in order to introduce the appropriate codons. The methods are well-known by the one skilled in the art. Of course, depending on the host, the genetic code may be adapted if needed or wished.

The present invention further relates to an expression cassette or construct, comprising a nucleic acid encoding a metallothionein tag as disclosed above or a polypeptide or protein comprising the metallothionein tags as disclosed above. The expression cassette or construct includes the necessary elements for expressing the metallothionein tag or the polypeptide or protein comprising the metallothionein tags in a convenient host cell and conditions.

The term “expression cassette” or “expression construct” refers to a nucleic acid construct comprising a regulatory region that is operatively linked to an expressible sequence. The term “expressible sequence” refers to a nucleotide sequence that can be transcribed to produce a functional product, including but not limited to a messenger RNA. Non-limiting examples of expressible sequence includes cDNA, genomic DNA, genomic DNA that comprises one or more exons that are transcribed, a gene sequence that contains one or more mutant nucleotide(s) which is not present in a naturally occurring sequence of the same gene. Typically, an “expression cassette” or “expression construct” comprises, or alternatively consists of one or more of (1) a polynucleotide encoding a metallothionein tag, optionally fused to a protein of interest, (2) a leader sequence, (3) a regulatory region, in particular a promoter region, and (4) a transcriptional terminator.

The term “operatively linked” indicates that the regulatory region and the expressible sequence are joined in the expression cassette or construct such that transcription of the expressible sequence is regulated by the regulatory region.

The regulatory region typically comprises a transcriptional promoter that is placed upstream at the 5′ end of the expressible sequence of interest at a distance which is effective for regulation of expression. It can further comprise enhancer or repressor element. The promoter may be for instance a strong or constitutive promoter such early or late SV40 promoters, CMV promoter. Alternatively, it can also be inducible promoter, such GAL4 promoter. A spacer may be present between the regulatory region and the expressible sequence. The sequence and length of the spacer can be determined by methods well known in the art.

The present invention also relates to an expression vector comprising a nucleic acid encoding a polypeptide or protein comprising the metallothionein tags as disclosed above or an expression cassette or construct as described above.

The vectors of the invention can be DNA or RNA, circular or linear, single- or double-stranded. Typically, it is a plasmid, phage, phagemid, virus, cosmid, artificial chromosome, etc. Exemplary viral vectors include adenovirus-derived vectors, adeno-associted viruses, herpes simplex derived vectors, hybrid adeno-associated/herpes simplex derived vectors, influenza viral vectors and alphaviruses. The vectors of the invention can comprise one or more of the following elements: (1) a functional self-replicating vector (including but not limited to, a shuttle vector, an expression vector, an integration vector, and/or a replication system), (2) a region for initiation of transcription (e.g., a promoter region, such as for example, a regulated or inducible promoter, a constitutive promoter), (3) a region for termination of transcription, (4) a leader sequence, and (5) a selectable marker. The vectors can be constructed by conventional molecular biology methods, well known to those skilled in the art, using for example restriction enzymes, ligation, cloning, replication, etc.

In a particular embodiment, the vector conveniently comprises a polylinker. By “polylinker” is intended a nucleic acid comprising at least two, and preferably three, four or more restriction sites for cleavage by one or more restriction enzymes. The restriction sites may be overlapping. Each restriction site is preferably five, six, seven, eight or more nucleotides in length. The polylinker can be between the regulatory region and the sequence encoding the metallothionein tag, at the 3′ end of the sequence encoding the metallothionein tag or flanking the sequence encoding the metallothionein tag (upstream and downstream of the coding sequence, at its 5′ and 3′ ends). The polylinker is suitable for introducing the sequence encoding the polypeptide or protein of interest in frame with the sequence encoding the metallothionein tag, in order to express a fusion protein including the metallothionein tag and the polypeptide or protein of interest.

Alternatively, the sequence encoding the metallothionein tag, optionally as a fusion protein with the protein of interest, can be inserted into the genome of a cell, for instance by homologous recombination, transposition or integrase mediated insertion. Accordingly, the nucleic acid or expression construct can be flanked by one or several recombination sites such as lambda recognition sequence (e.g., attL or attB), Cre-Lox, FLP recombination site, and the like.

Preferably, the vector provides a transient expression of the metallothionein tag in the recombinant cell. In this embodiment, a cell is transfected or transformed with the vector and cultivating under conditions suitable for the protein expression. In this embodiment, the vector is selected to be unstable inside the cell; thereby the expression is lost after a relatively short period.

The present invention relates to host cell or recombinant cell comprising a nucleic acid, an expression cassette or construct, or an expression vector as disclosed above. The host cell can be a prokaryotic cell such as E; coli or an eukaryotic cell, such as a mammalian cell.

Kits

The present invention relates to a kit comprising a nucleic acid, an expression cassette or construct, or a vector as disclosed above. It can further include a manufacturer notice with instructions. Optionally, it can comprise the lanthanide to be used in combination with the metallothionein tag of the invention, preferably selected from the group consisting of Eu, Sm, Tb, and Dy, more preferably Eu. Optionally, the kit may comprise other reagents such as restriction enzymes, in particular those corresponding to the polylinker flanking the sequence encoding the metallothionein tag.

The present invention also relates to the use of the kit for microscopy studies or analysis and/or for sample preparation suitable for microscopy studies or analysis. More generally, the present invention relates to the use of a metallothionein tag of the invention, a protein fusion comprising at least one metallothionein tag and a protein of interest, a complex lanthanide-metallothionein tag or fusion protein, a nucleic acid, expression construct or vector encoding them, a recombinant cell comprising such a nucleic acid, expression construct or vector for microscopy studies or analysis and/or for sample preparation suitable for microscopy studies or analysis. More particularly, the microscopy studies or analysis are electron microscopy (SEM and TEM) and/or light microscopy, more preferably both electron and light microscopies (CLEM).

Methods

The present invention relates to methods for examining a protein of interest by microscopy, preferably by electron and/or light microscopy, more preferably by both electron and light microscopy (e.g., CLEM), the method comprising
providing a sample comprising a fusion protein of the metallothionein tags of the invention with a protein of interest,
contacting the fusion protein with lanthanides,
then, observing the lanthanides by microscopy, preferably by electron and/or light microscopy, more preferably by both electron and light microscopy (e.g., CLEM).

The present invention relates to a method for analyzing a protein of interest by light and/or electron microscopy, comprising
a. providing a sample comprising a fusion protein comprising at least one metallothionein tag of the invention and the protein of interest;
b. contacting the sample with lanthanide(s); and,
c. analyzing the sample by light and/or electron microscopy.

Preferably, the fusion protein is present in a cell. In particular, the step a) comprises providing a cell comprising a nucleic acid, expression construct or expression vector encoding the fusion protein, and cultivating the cell under conditions allowing the expression of the fusion protein.

More particularly, the present invention relates to a method for labeling a protein of interest in a cell without disrupting the cellular membrane of the cell, comprising
a. providing a cell comprising an expression cassette or construct encoding a fusion protein comprising at least one metallothionein tag of the invention and the protein of interest;
b. cultivating the cell in conditions allowing the expression of the fusion protein;
c. contacting the cell with lanthanide(s); and
d. optionally, analyzing the sample by light and/or electron microscopy.

In the above-mentioned methods, the steps of cultivating the cell under conditions allowing the expression of the fusion protein and of contacting the sample with lanthanide(s) can be performed sequentially and simultaneously. Optionally, the method comprises, prior the step of analyzing the sample by light and/or electron microscopy, a step of preparing the sample for being suitable for microscopy analysis. In particular, this step may include a step of fixing the sample. The sample may be fixed by chemical fixation or freezing. In addition, the preparation step may further comprise a step of slicing the sample into thin sections of a thickness in the range of about 25 nm to 1 μm or direct observation in cyro-electron microscopy.

In the above-mentioned methods, the lanthanide is selected from the group consisting of Eu (Europium), Terbium (Tb), Samarium (Sm) and Dysprosium (Dy), preferably Eu (Europium). In a most preferred embodiment of the methods, the sample is analyzed by both light and electron microscopies (CLEM).

The sample is preferably cells, in particular prokaryotic or eukaryotic cells, or tissue. In particular, prokaryotic cells can be bacteria and eukaryotic cells can be a fungal cell such as yeast, animal, mammalian, human, or plant cells. Eukaryotic cell can be for example fibroblast, hematopoietic, endothelial and epithelial cell. Cell can be derived from a healthy or pathologic tissue or organism. Cell can be wild type or modified/recombinant cells. In a particular embodiment, the mammalian cell can be a tumor cell. In particular, the cells may provide from a cell culture or may have been isolated from a tissue or an organism.

The present invention is further illustrated, without being limited to, by the above examples.

### EXAMPLES

Creating a LM and TEM Correlative Probe

The inventors have tested the use of Eu combined with clonable MTtag for correlative imaging. Thus, they have transformed E. coli K12 with MTtag and grow it in presence of EuCl3.

Eu Toxicity

The inventors were first interested in evaluating Eu toxicity in their working conditions. To this purpose, they have submitted E. coli to a serial of growing tests called Minimal Inhibitory Concentration (MIC). This test aimed to determine the lowest concentration able to inhibit the bacteria growth. It consisted in growing bacteria in presence of several concentrations of the tested compound.

Serial dilution series of the substance to be tested were inoculated with bacteria at 0.3 D.O_{550 nm} diluted 1/16000 in culture medium. D.O_{550 nm} was followed until stationary phase. Minimal concentration of the test substance preventing stationary phase was considered as MIC.

For the Eu MIC determination, the inventors have tested EuCl3 concentrations between 100 μM and 2 mM. E. coli K-12 and pBAD arabinose inducible MTtag transformed E. coli K-12 were tested. E. coli K12 were transformed by electroporation with the pBAD plasmid down control of BAD promoter (Guzman, L. M., et al. J Bacteriol 177, 4121-4130 (1995); Khlebnikov, A. & Keasling, J. D. Biotechnol. Prog. 18, 672-674 (2002)). Bacteria pre-cultures were produced at 37° C. in Luria-Bertani (LB) medium with added ampicillin (100 g/ml) in transformed strain. The pre-cultures were used to inoculate tubes of minimum media M9 (with or without ampicillin) until exponential phase D.O_{550 nm}≈0.3. Then, 1/16000 dilutions were prepared in M9 medium (with or without ampicillin). 1 μl of the diluted bacteria were added to 200 μl of M9-EuCl3 at different concentrations in 96 wells plates. Bacterial grow was followed until stationary phase. The D.O_{550 nm} was measured on a Tecan Infinite 200 PRO.

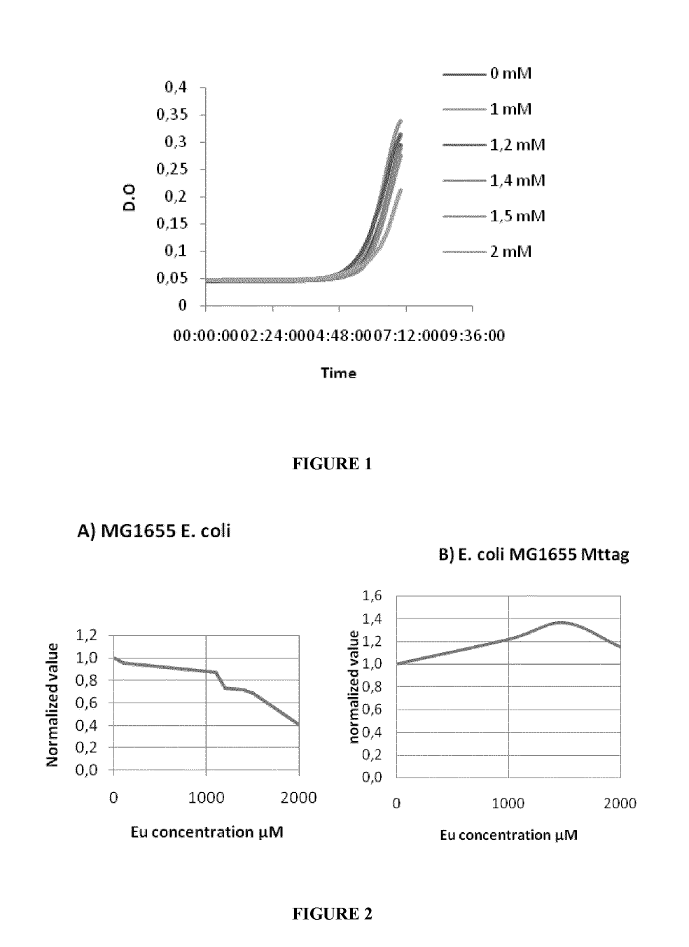

Interestingly, MIC test (FIG. 1) shown that bacteria is viable until EuCl₃ concentrations higher than 2 mM. The test was repeated 10 times with equivalent results. Growing slow down is observed after 1 mM EuCl₃ concentration, but it does not impact bacterial viability as they are able to grow in LB plates when plating. The maximal EuCl₃ concentration tested correspond to the maximum possible as the molecule precipitates at higher concentrations indicating that EuCl₃ is not toxic for E. coli K-12 transformed or not. Interestingly, bacteria expressing MTtag grow faster than non-induced and wild-type E. coli K12 when EuCl₃ is present (FIG. 2). This can be explained by MT properties as primary biological agent involved in the homeostatic regulation of essential metals on the cells or by its contribution to cellular antioxidant functions. These results suggest an interaction between MTtag and EuCl₃ within the cell. In order to evaluate this hypothesis, the inventors assayed to determine if Eu clusters can be formed after MT-tag induction.

MT is able to form Europium Clusters in Bacterial Cytoplasm

E. coli K12 expressing MTtag were grown with 200 μM EuCl₃, until 0.3 D.O_{550 nm} and then induced with 0.3 mM IpTG around 2.5 hours. Cells were fix by Karnovsky method (Karnovsky, M. J Journal of Cell Biology 27, 137A-138A (1965)) and embedded by gradual spur resin inclusion. Sections of 50-100 nm were deposed on 300 mesh Quantifoil holey carbon grids (R 3.5/1 Cu/Rh, Quantifoil Micro Tools, Jena, Germany) before observation by STEM (Scanning Transmission Electron Microscopy) and TEM modes. Electron microscopy imaging was carried out on a field emission gun transmission electron microscope JEOL-2200FS, equipped with an in-column energy filter (Omega Filter). Microscope was operated at 200 KV. Transmission images were recorded on a Gatan ssCCD 2k×2k camera at variable magnifications (from 8 to 10×). STEM images (512*512 px) were acquired using a Hamamatchu CCD at nominal magnifications of 200.000× employing an HAADF-STEM detector. Electron energy loss spectra were acquired with the 200 μm condenser aperture and spot size 5 by cumulating 1 second spectra. EL/P Gatan Digital Micropraph software has been used for spectrum acquisition.

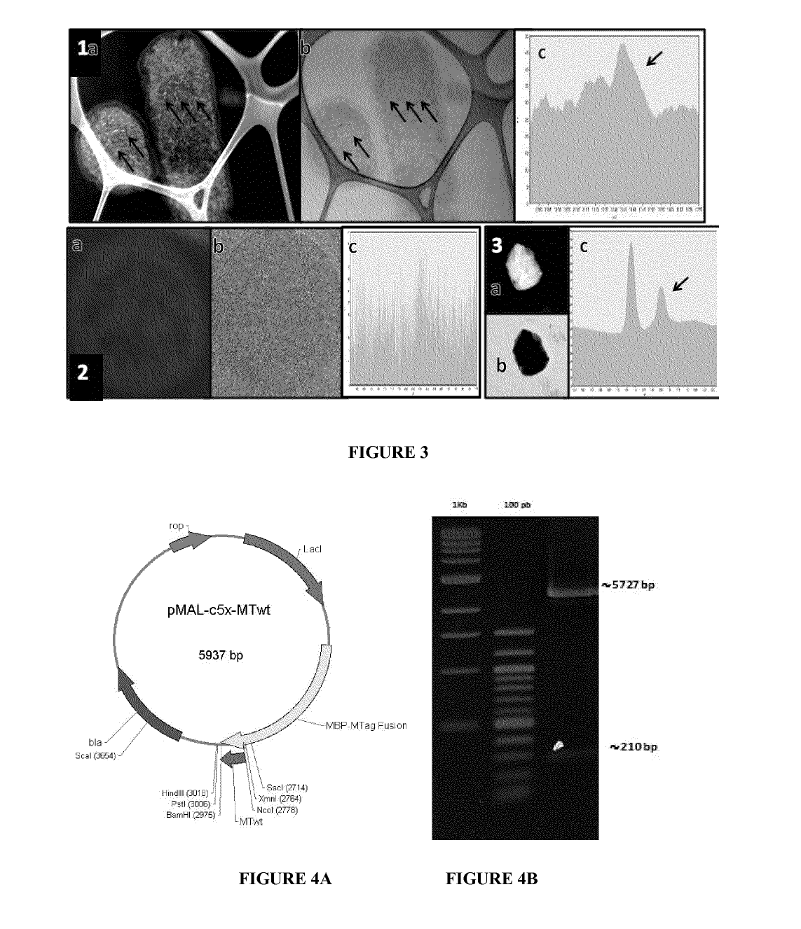

HAADF (FIG. 3-1a), and BF (FIG. 3-1b) STEM images; shows electron dense nanometric clusters in the cytoplasm of bacteria expressing MT-tag after incubation with EuCl₃. EELS performed on these samples (FIG. 3-1C) indicate that these clusters are composed by Eu. These clusters are observable by Z-contrast imaging in STEM-HAADF mode only in bacteria expressing MTtags and incubated in presence of EuCl₃, but clusters aren't observed in non transformed bacteria (FIGS. 3-2a,b,c). These experiences show that MTtag is able to capture Eu in vivo and to form nano-metric clusters. Therefore, combining MTtags with lanthanides can be an interesting approach for nanometric tracking of biological macromolecules by different imaging methods and mainly for CLEM (Correlative Light and Electron Microscopy). Thus, the inventors have evaluated the potential fluorescence of Eu bound to MT-tags.

Antenna is Required for MTtag-Eu³ Fluorescence

In order to evaluate the capacities of MT-tagEu for CLEM, the inventors decided to perform in vitro studies with the isolated protein. MTtag was cloned in the commercial vectors pMAL™-c5X (FIG. 4) and fused to Maltose binding protein. The fusion MBP-MTtag was expressed as a soluble protein in the cytoplasm, allowing purification.

The protein purification started with cultures from single colonies LB Ampicillin 100 μg/ml, leaving them over night at 37° C. with aeration. 250 ml growth cultures were inoculated with 5 ml of the overnight culture and were grown adding 1% glucose until an O.D₅₅₀=0.5. Protein production was induced with 0.3 mM IPTG. Then, cells were pelleted at 4000×g. and were placed in 50 ml falcon tubes. Cells were resuspended on Column buffer (20 mM Tris-HCl, 200 mM NaCl, 1 mM EDTA). Cells suspensions were lysed by 3 sonication in cycles of pulses of 10 sec. Lysis was monitored maintaining the protein concentration constant, measuring them with DC™ Protein Assay system. Once lysed, cell suspensions were pellet at 9000×g for 20 min. The resulting supernatants were diluted in wash buffer (20 mM Tris-HCl) to 100 ml and then loaded onto a pre-equilibrated 1.5 ml amylose column (New England Biolabs). The New England Biolabs protocol suggests the use of a column buffer supplement with β-mercaptoethanol or other reducing agents. However, MT-tag need form thiol structures, contributing to bind Eu. This is the reason why the inventors have modified column buffer composition, to conserve the affinity-binding domain of MT-tag. Thus, β-mercaptoethanol or other reducing agents were not added to buffer solutions. After loading supernatant into column, it was washed with 10 column volumes using wash buffer. Bounded protein was eluted in 0.5 ml fractions of wash buffer supplemented with 10 mM maltose (Sigma Chemical Corp). Residual EDTA was removed by dialysis with wash buffer during 36 h.

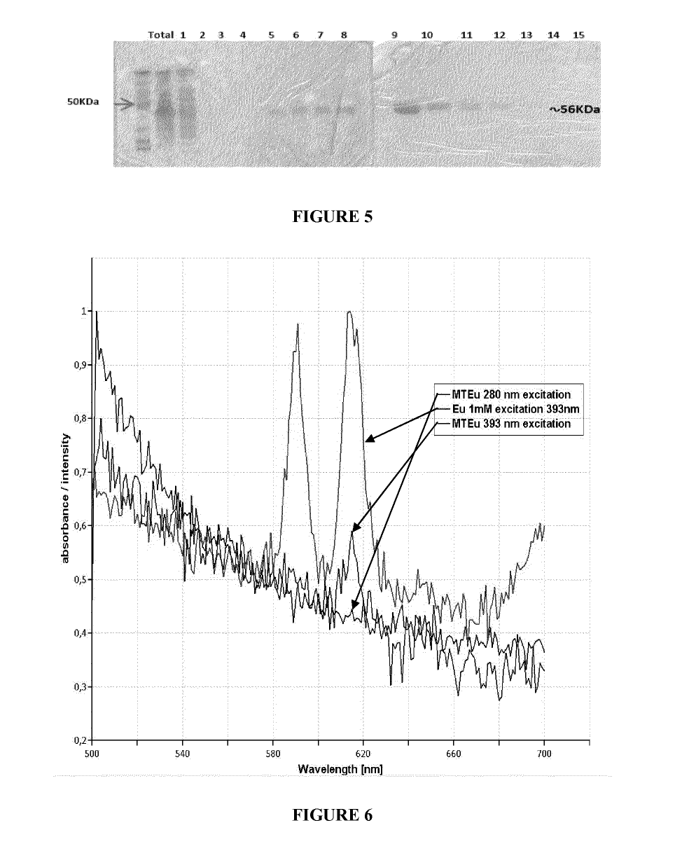

Protein concentrations of eluted fractions were quantified by Bradford Protein Assay. Typically, eluted peak fractions were found to have concentrations MT-MBP of about ≈5-6 mg/ml. Aliquots (100 μl) were fast frozen in liquid nitrogen and stored at −78° C. (FIG. 5).

Once protein isolated, the fluorescence of MTtag-Eu³ complex was tested as follows: MTtag purified protein was mixed with EuCl₃ 1 mM by 5-10 minutes and then dialyzed on column buffer (0.02 M Tris-HCl, pH 7.4) to form MTtag-Eu³ complex. 50 nM of MTtag-Eu 3³⁰ were used for fluorescence measurements performed by using a Spectrofluorimeter (Jobin Yvon, France). The spectrofluorimeter was operated at 280 nm excitation wavelength. The spectra were corrected for emission intensity by using manufacturer-supplied correction factors. Slit widths were 5 nm for excitation and emission. A band pass filter 370 L to avoid interference from harmonic doubling was used. As positive control a 2 mM solution of EuCl₃ was used to calibrate the instrument. 393 nm was used as excitation wavelength because it is specific for this element. Specific Eu signal to 585 nm and 612 nm of emission are expected.

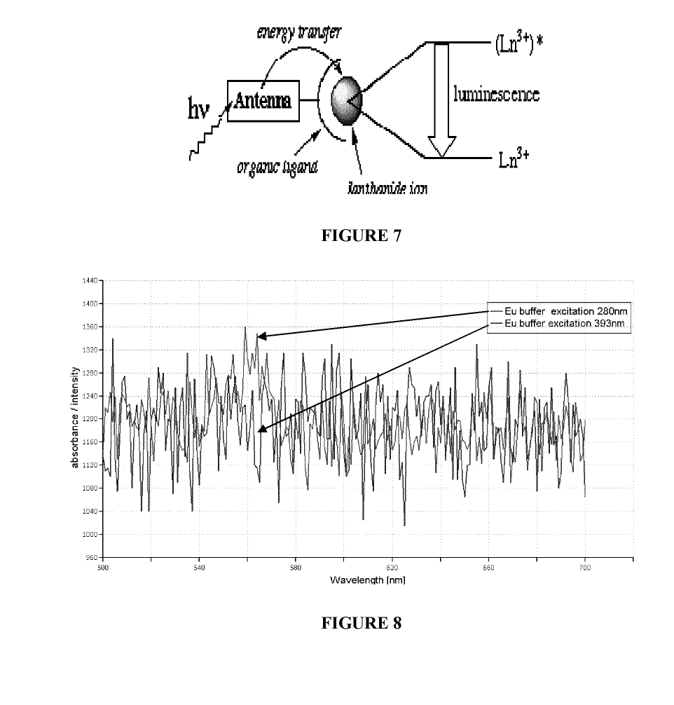

The excitation wavelength optimal for organic compounds depends on their own characteristics. Therefore, the excitation energy for the new MT-tagEu complex should be determined. The first test to determine excitation energy of the complex MT-tagEu was performed using a scanning excitation, with a wavelength emission of 612 nm. As expected, results reveal a total absence of fluorescence for MTtag, but also no signal is found by MTtag-Eu³⁺ complex, because the peptide sequence haven't Trp, Tyr and Phe able to transfer energy to the Eu³⁺ (FIG. 6). However, the inventors observed a signal characteristic of Eu³⁺ when MTtag-Eu³⁺ complex is excited at 393 nm. This result suggests that MTtag had capture Eu³⁺ and prevented his elimination during dialysis.

Test was repeated in vivo in E. coli K12 expressing MTtag. Bacteria were grown with 200 μM EuCl₃, until D.O_{550 nm} 0.3 and then induced with 0.3 mM IpTG around 36 hours. In conclusion, both, in vivo and in vitro tests show that MTtag does not provide sufficient activation energy to increase Eu fluorescence. These results also suggest that Eu complexed by MTtag cannot receive transference energy of other neighbor molecules.

These results can be explained by different reasons: (i) Eu is weakly fluorescent, (ii) europium concentration on MTtag domain is necessarily lower than 10 mM, making detection impossible without fluorescence amplification, (iii) the absence of resonance structures on affinity domain of MTtag enabling the energy transfer to Eu⁺³.

Then, the inventors had focused study in the way to modify MTtag to develop an antenna in the protein MT to improve the Eu³⁺ fluorescence.

Modifying MTtag as an Antenna to Improve Eu³⁺ Fluorescence

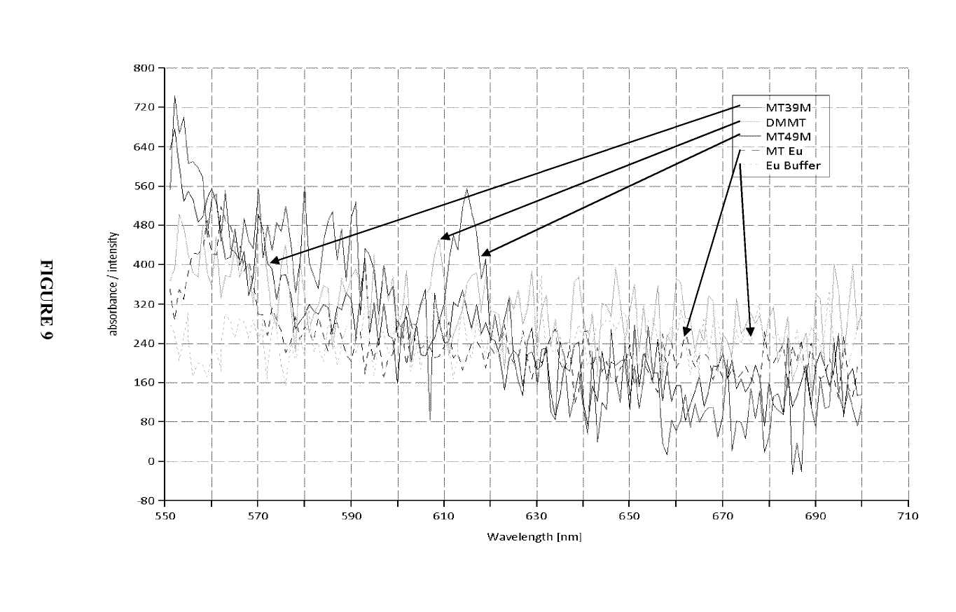

In the organic chromophores, the forbidden nature of the f-f transitions is also reflected in low extinction coefficients, making direct photoexcitation of Lanthanides ions difficult. The difficulty has overcome by using energy transfer from organic chromophores to lanthanide ions. This organic complex absorbs light and transfers energy to the lanthanide ions that change it into “excited state”, with the following consequence; the ions relax to their fundamental state and emit light as well (FIG. 7).

The energy transfer can proceed via dipolar or multipolar interactions between sensitizer and acceptor (lanthanide ion). These two different mechanisms have been described by Förster and Dexter as mechanism (Malta et al. Biochemistry 46, 9080-9088 (2007)).

Previous studies (Hong et al, 2005, Protein Engineering, Design & Selection: PEDS, 18(6), 255-263) have described the use of Tip for FRET between metal and ligands binding in MT-2 isoform. The present work inspired in this, using punctual mutations replacing Cys by Trp in the a domain of MT-1.

For Maret W. group, these mutations did not intended to produce an antenna, they tried to found a MT FRET sensor to check formation and disassembly of the metal cluster on MT domain. For this reason, the new mutations are different. Mainly, they were focused on improving sensitivity in energy transfer.

The antenna must be as close as possible to the affinity domain of europium domain, i.e. the antenna must be contained in the sequence of the protein domain. The natural aromatic amino acids that can be donor at a higher excitation wavelength are Trp (excitation: 280 nm and emission 300-450 nm).

MT is a small protein of 60 amino acids of which 20 are Cys bounding seven Zn atoms in two Zn thio late clusters. Therefore, none of these Cys can be chemically modified without severely perturbing or destroying the structure of the clusters and the ability of the protein to fix metals, and then lanthanides. Based on this, The inventors decided to introduce Trp as close as possible to Cys groups by replacing an amino acid without effect on the activity of the protein. To achieve this purpose two approaches are possible:
(i) Introduce a punctual mutation replacing an amino acid neighbor of a Cys by Trp. In this case, Trp is able to FRET with the neighbor Cys and with bound lanthaninde particle;
(ii) Replacing the amino acid close to a Cys, but not enough to be it neighbor. FRET remains possible between Tip and bound particle.

The inventors worked with a conserved MT consensus sequence, very similar to the one described by Mercogliano et al. 2007 (Journal of Structural Biology, 160(1), 70-82), or Diestra et al 2009 (Journal of Structural Biology, 165(3), .157-168). In silico assays performed with PyMol program (DeLano W. L The PyMOL Molecular Graphics System. (DeLano Scientific: San Carlos, Calif., USA,). www.pymol.org) provided them with interactive visualization of MT and a preliminary molecular modeling that permitted the selection 2 candidates as possible mutants. The criteria of choice of the candidates were based on the work of Malta et al. (Biochemistry 46, 9080-9088 (2007)) who performed theoretical calculations on the sensitization of Eu³⁺, Yb³⁺, and Sm³⁺ by sensitizers. They derived formulas for dipolar and multipolar energy transfer (Förster type) and for exchange mechanism (Dexter type). For energy transfer from an excited singlet or triplet state to the (2S+1) FJ levels of lanthanide ions, the selection rules for energy transfer are:

|ΔJ|32 0, 1 (J=J′=0 excluded) for a Dexter mechanism

|ΔJ|=2, 4, 6 for a Förster mechanism.

And the selection criteria were:
(i) the energy gap between donor (sensitizer singlet or triplet state) and acceptor (lanthanide ion levels) should not be too large,
(ii) the selection rules outlined above, and
(iii) Significant matrix elements of the orbital overlap.

Following these criteria, the selected positions for mutations are shown below in bold:

(SEQ ID No 3)
1-MDPNCSCSTG GSCTCTSSCA CKNCKCTSCK KSCCSCCPVG
CSKCAQGCVC KGAADKCTCC A-61

Before genetically completion of mutants, 3D simulations with the program VMD (Visual Molecular Dynamics) were performed. For this, a data base .pfs (protein structure file) was created in collaboration with Liliane Mouawad Institut Curie/INSERM U759, based on a topology file of MT consensus binding Ca²⁺, but including the Eu³⁺ parameters (mass, charge, van der Waals). This permitted determination of the relative distances between Eu ions and the two types of antenna model. In 39V mutation, the distance between Eu and antenna W was 8.72 A° . In the second case, 49V distance was 9.68 A. Both distances are in the range (5-10 A°) required to produce energy transfer. Based on these simulations, the inventors decided to produce 3 mutants: V39W (MT39), V49W (MT49) and the double mutant V39W/V49W (MTDM).

MT tags purified protein was mixed with EuCl₃ 200 mM by 5-10 minutes and then dialyzed on washing buffer (0.02 M Tris-HCl, pH 7.4) during 1 day. 50 nM of MT tags protein on column buffer has been tested. Fluorescence measure in MTtag-Eu³⁺ wt (negative control) and MTtag mutants MT39, MT49 and MTDM. As positive control, a EuCl₃ 2 mM was exited at 393 nm wavelength, to calibrate the instrument. As negative control, purified protein without Eu and a washing buffer containing EuCl₃ 200 mM has been dialyzed on washing buffer (0.02 M Tris-HCl, pH 7.4) by 1 day.

A spectrofluorimeter Fluoromax (Jobin Yvon, France) operated at Trp excitation wavelength 280 nm. The spectra were corrected for emission intensity by using manufacturer-supplied correction factors. Slit widths were 5 nm for excitation and emission slits. A band pass filter 370 L to avoid interference from harmonic doubling was used.

As expected, negative controls did not emission. The dialyzed Eu buffer did not fluorescence at 280 nm excitation wavelength, as there is no interaction between Eu and Trp. But also no fluorescence is detected at 393 nm wavelength because dialyze process eliminates Eu. It shows that Eu in sample is only present when a binding-structure saves it (FIG. 8).

Emission spectrums for the different MTs label are shown in FIG. 9. MTDMEu³⁺ and MT39Eu³⁺ present a lower signal, almost imperceptible. But in any case, these small signals were higher than their controls without Eu. Also, the signal seems to be most specific for MTDM respect to MT39 in this order.

A much more promising result was obtained with the mutant MT49Eu. A fluorescence signal was found in it. In addition, its negative controls MT-49M without Eu gave no signal. The luminescence intensity for its new MT mutant label was weakly compared to other typical organic fluorophores. However, it first suggests a good beginning by future improvements.

Electron Microscopy

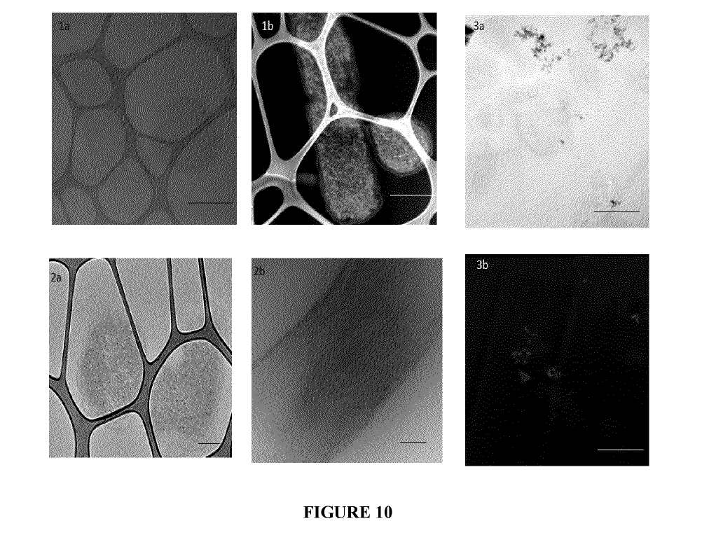

FIG. 10-1 shows that mutant MT49 is able to form Eu nanometric clusters as wild type form. Clusters are perceptible in HAABF and DF STEM mode, showed with arrows. But, it does not happen in FIG. 10-3, the negative control where the bacteria cells in absence of protein expression is unable to form nanocluster.

1-15. (canceled)

16. A metallothionein tag comprising a metallothionein or a fragment thereof of at least 60 amino acids including the cysteine-rich domain, wherein the metallothionein tag has at least one substitution by a tryptophan residue of an amino acid directly adjacent to a cysteine residue.

17. The metallothionein tag according to claim 16, wherein the metallothionein tag comprises or consists of the sequence of SEQ ID No 1, 2 or 3 and has at least one substitution by a tryptophan residue of an amino acid in a position selected from the group consisting of 4, 6, 8, 12, 14, 16, 18, 20, 22, 23, 25, 27, 28, 30, 32, 35, 38, 40, 42, 43, 45, 47, 49, 51, 56, 58, and 61.

18. The metallothionein tag according to claim 16, wherein the metallothionein tag comprises or consists of the sequence of SEQ ID No 1, 2 or 3 and has at least one substitution by a tryptophan residue of an amino acid in a position selected from the group consisting of 6, 14, 20, 25, 35, 49, and 58.

19. The metallothionein tag according to claim 17, wherein the residue in position 49 has been substituted by a tryptophan residue and, optionally one or several amino acids in positions 4, 6, 8, 12, 14, 16, 18, 20, 22, 23, 25, 27, 28, 30, 32, 35, 38, 40, 42, 43, 45, 47, 51, 56, 58 and 61.

20. The metallothionein tag according to claim 16, wherein the metallothionein tag has at least one substitution by a tryptophan residue of an amino acid surrounded by two cysteine residues.

21. A protein or polypeptide comprising at least one metallothionein tag according to claim 16 and a protein of interest as a fusion protein.

22. A protein-lanthanide complex comprising:
a) a metallothionein tag according to claim 16; and
b) a lanthanide bound to the metallothionein tag.

23. The protein-lanthanide complex according to claim 22, wherein said lanthanide is selected from the group consisting of Eu (Europium), Terbium (Tb), Samarium (Sm), and Dysprosium (Dy).

24. A nucleic acid, expression construct or expression vector encoding a metallothionein tag according to claim 16 and optionally further comprising a polylinker upstream or downstream of a nucleic acid encoding a metallothionein tag.

25. A recombinant cell comprising the nucleic acid, expression construct or expression vector according to claim 24.

26. A kit comprising the nucleic acid, expression construct or expression vector according to claim 24 and optionally other reagents such as a lanthanide and/or a restriction enzyme.

27. A method for analyzing a protein of interest by light and/or electron microscopy, comprising:
a) providing a sample comprising a fusion protein according to claim 21;
b) contacting the sample with a lanthanide; and
c) analyzing the sample by light and/or electron microscopy.

28. The method according to claim 27, wherein the fusion protein is present in a cell.

29. The method according to claim 28, wherein step a) comprises providing a cell comprising a nucleic acid, expression construct or expression vector encoding a fusion protein and cultivating the cell under conditions allowing the expression of the fusion protein.

30. The method according to claim 27, wherein the lanthanide is selected from the group consisting of Eu (Europium), Terbium (Tb), Samarium (Sm), and Dysprosium (Dy).

31. The method according to claim 27, wherein the sample is prepared for being suitable for microscopy analysis prior to the analyzing step.

32. A protein-lanthanide complex comprising a polypeptide according to claim 21 and a lanthanide bound to the metallothionein tag.

33. The polypeptide-lanthanide complex according to claim 32, wherein said lanthanide is selected from the group consisting of Eu (Europium), Terbium (Tb), Samarium (Sm), and Dysprosium (Dy).

34. A nucleic acid, expression construct or expression vector encoding a protein or polypeptide according to claim 21, and optionally further comprising a polylinker upstream or downstream of a nucleic acid encoding a metallothionein tag.


SEQUENCE LISTING (4 sequences)

SEQ ID NO: 1 - protein, 61 aa, artificial
  Metallothionein consensus sequence
XDXXCXCXXXXSCXCXXSCXCKXCXCXSCKKXCCXCCPXGCXXCAQGCXCKXXXXKCXCCX

SEQ ID NO: 2 - protein, 61 aa, artificial
  Metallothionein consensus sequence
XDPNCSCXTGGSCTCXXSCXCKXCKCTSCKKSCCSCCPVGCXKCAQGCXCKGAXXKCXCCX

SEQ ID NO: 3 - protein, 61 aa, artificial
  Metallothionein consensus sequence
MDPNCSCSTGGSCTCTSSCACKNCKCTSCKKSCCSCCPVGCSKCAQGCVCKGAADKCTCCA

SEQ ID NO: 4 - DNA, 192 bp, artificial
  Sequence encoding SEQ ID No 3
atggacccgaattgtacgtgtagcaccggtggtagctgtacctgtaccagctcttgtgcatgtaaaaattgtaaatgcaccagctgcaaaaaaagctgttgtagctgttgcccggttggttgtagcaaatgtgcacagggttgtgtttgtaaaggtgcagcagataaatgtacctgttgtgcaggatcctaa